(19) United States
(12) Patent Application Publication
(10) Pub. No.: US 2018/0368969 A1
(43) Pub. Date: Dec. 27, 2018

(54) Tendon Reinforcement for Rotator Cuff Repair

(71) Applicant: Drexel University, Philadelphia, PA (US)

(72) Inventors: Desiree Martini, Mayfield, PA (US); Michael Chen, Blue Bell, PA (US); David Christopher Rodak, Warrington, PA (US); Kenneth Giovanni Rodriguez, Jackson, NJ (US); Kara L. Spiller, Philadelphia, PA (US)

(73) Assignee: Drexel University, Philadelphia, PA (US)

(21) Appl. No.: 15/992,222

(22) Filed: May 30, 2018

Related U.S. Application Data
(60) Provisional application No. 62/512,197, filed on May 30, 2017.

(51) Int. Cl. A61F 2/08; A61F 2/40

(52) U.S. Cl. CPC A61F 2/0811; A61B 2090/0815; A61F 2/40

(57) ABSTRACT

A tendon reinforcement assembly includes a rivet having a female portion having a female free end, a female connecting end, and a female portion axial passage extending therethrough. A male portion has a male free end, a male connecting end adapted to be inserted into the female free end, and a male portion axial passage extending therethrough. The male portion axial passage extends coaxially with the female portion axial passage. A suture extends through the male portion axial passage and the female portion axial passage. The suture has a first suture end extending outwardly of the male free end and a second suture end extending outwardly of the female free end.

### CROSS-REFERENCE TO RELATED APPLICATION

The present application claims the benefit of U.S. Provisional Patent Application Ser. No. 62/512,197, filed on May 30, 2018, which is incorporated herein by reference in its entirety.

### BACKGROUND OF THE INVENTION

### Field of the Invention

The present invention relates to reinforcement devices that are used during the repair of rotator cuff injuries.

### Description of the Related Art

Rotator cuff/supraspinatus tendon tears are common injuries, with over 80,000 surgeries performed annually in the U.S. However, re-tears following surgery are extremely common, occurring in over 82% of repairs. The main failure mode for re-tears involves sutures tearing through tendon at the suture-tendon interface.

It would be beneficial to provide a reinforcement device to reduce the likelihood of re-tears.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a tendon reinforcement assembly comprising a rivet having a female portion having a female free end, a female connecting end, and a female portion axial passage extending therethrough. A male portion has a male free end, a male connecting end adapted to be inserted into the female free end, and a male portion axial passage extending therethrough. The male portion axial passage extends coaxially with the female portion axial passage. A suture extends through the male portion axial passage and the female portion axial passage. The suture has a first suture end extending outwardly of the male free end and a second suture end extending outwardly of the female free end.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a front view of a rivet assembly according to an exemplary embodiment of the present invention;

FIG. 2 is a top plan view of the rivet assembly of FIG. 1;

FIG. 3 is a side elevational view of the rivet assembly of FIG. 3;

FIG. 4 is an exploded perspective view of the rivet assembly of FIG. 1;

FIG. 5A is a side elevational view of the rivet assembly of FIG. 1 in a first ultimate tensile stress (“UTS”) sample;

FIG. 5B is a side elevational view of the rivet assembly of FIG. 1 in a second ultimate tensile stress (“UTS”) sample;

FIG. 5C is a side elevational view of the rivet assembly of FIG. 1 in a third ultimate tensile stress (“UTS”) sample;

FIG. 6 is a graph of Compressive Stress v. Rivet Diameter for the rivet assembly of FIG. 1;

FIG. 7 is a graph of Tendon Stress X 2 v. Rivet Diameter for the rivet assembly of FIG. 1;

FIG. 8 is the graph of FIG. 6 overlaid onto the graph of FIG. 7;

FIG. 9 is a sectional view of a top portion of the rivet assembly of FIG. 1;

FIG. 10 is a sectional view of a top portion of a rivet assembly according to an alternative exemplary embodiment of the present invention;

FIG. 11 is a graph showing Stress v. Strain for different diameter rivets used in the inventive rivet assembly; and

FIG. 12 shows a gap being formed in a tendon that is pulled by the assembly of the present invention.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

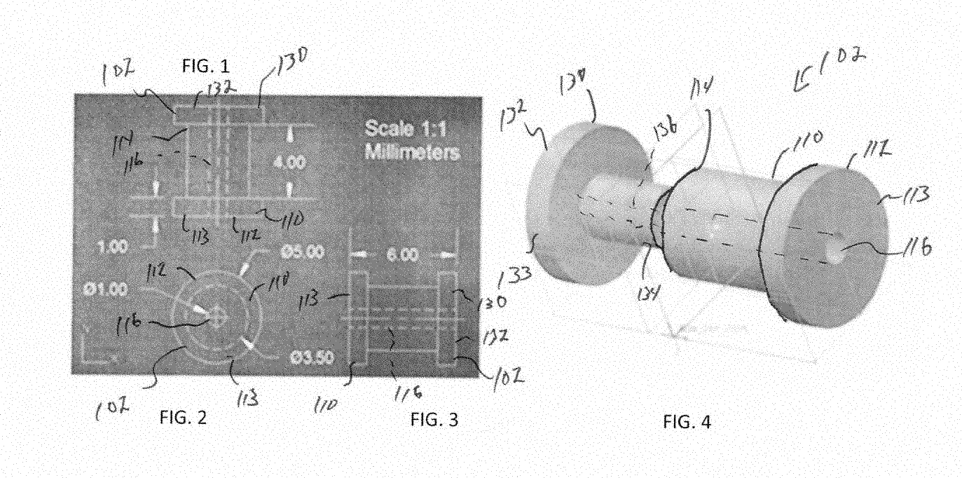

Referring to FIGS. 1-5C and 9, a tendon reinforcement assembly 100 (“assembly 100”) according to an exemplary embodiment of the present invention is shown. Assembly 100 includes a rivet 102 and suture 150 that is used to repair rotator cuff/supraspinatus tendon repairs in humans, although those skilled in the art will recognize that assembly 100 can be used for other repairs as well.

Assembly 100, which can include a hollow rivet 102 having various diameters, is placed at suture/tendon interfaces and sutures are passed through assembly 100 rather than just through tendon as per traditional repairs. Sutures 150 are threaded through rivet 102 rather than directly through tendon. The purposes of the rivet 102 are to improve suture force distribution and to provide a protective barrier between the suture 150 and the tendon.

Rivet 102 has two main purposes: to distribute load more evenly to a tendon; and to provide a mechanical barrier between suture and tendon. Since the diameter of river 102 is greater than that of a single suture, the suture tension transferred to tendon per unit area (stress) is decreased compared to that of a single suture alone. Rivet 102 also provides a mechanical barrier to delay wear and tear of the suture through the tendon, therefore improving fatigue strength with repeated loading. In an exemplary embodiment, rivet 102 is about 6 millimeters in length.

Referring specifically to FIGS. 1-4 and 9, assembly 100 includes a female portion 110 and a male portion 130 that is inserted into the female portion 110. The female portion 110 has a female free end 112 having a flange 113, a female connecting end 114, and a female portion axial passage 116 extending therethrough. In an exemplary embodiment, the female portion 110 can be about 4 millimeters in length and the flange 113 can be about 1 millimeter thick and about 5 millimeters in diameter.

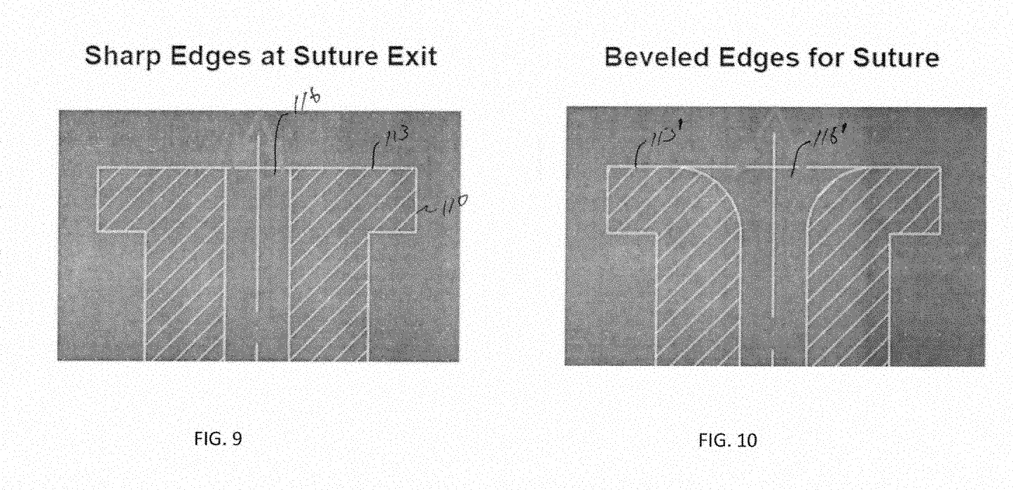

In an exemplary embodiment, shown in FIG. 9, a transition between axial passage 116 and flange 113 can be generally sharp. In an alternative embodiment, shown in FIG. 10, the transition between an axial passage 116′ and a flange 113′ can be curved, or beveled, to reduce the effect of a suture rubbing against the transition and being cut.

Similarly, the male portion 130 has a male free end 132 having a flange 133, a male connecting end 134 adapted to be inserted into the female free end 114, and a male portion axial passage 136 extending therethrough. The male portion axial passage 136 has a diameter of about 1 millimeter. In an exemplary embodiment, the flange 133 can be about 1 millimeter thick and about 5 millimeters in diameter.

The male portion axial passage 136 extends coaxially with the female portion axial passage 116. Similar to the transition between axial passage 116 and flange 113, the transition between axial passage 136 and flange 133 can be sharp or beveled.

Materials used for female portion 110 and male portion 130 must not elicit immune or foreign-body responses since the device is destined for in vivo use. In an exemplary embodiment, female portion 110 and male portion 130 are constructed from 316 stainless steel. Alternatively, female portion 110 and male portion 130 can be constructed from UHMWPE due to its low friction properties. Those skilled in the art, however, will recognize that female portion 110 and male portion 130 can be constructed from other biocompatible materials. Thus, materials used must perform under physiological conditions including temperature (about 37° C.), pH (between about 7.0- and about 7.5), enzymatic activity, and humidity/hydration (about 65% water).

A suture 150 extends through the male portion axial passage 136 and the female portion axial passage 116. The suture 150 has a first suture end 152 extending outwardly of the male free end 132 and a second suture end 154 extending outwardly of the female free end 112.

Rivet 102 is designed to reduce the likelihood of tearing through tendon after insertion. If rivet 102 has too small of an outer diameter, the concentration of forces may make it likely to tear through the tendon if applied forces on the assembly are too great or too extensive. Alternatively, if assembly 100 has too large of an outer diameter, an exceedingly large hole has to be formed in the tendon, which can result in subsequent tendon tears.

Stress on rivet 102 is inversely proportional to the diameter of rivet 102. As the diameter of rivet 102 increases, the compressive stress exerted by rivet 102 on the tendon tissue decreases, making suture tear-through less likely.

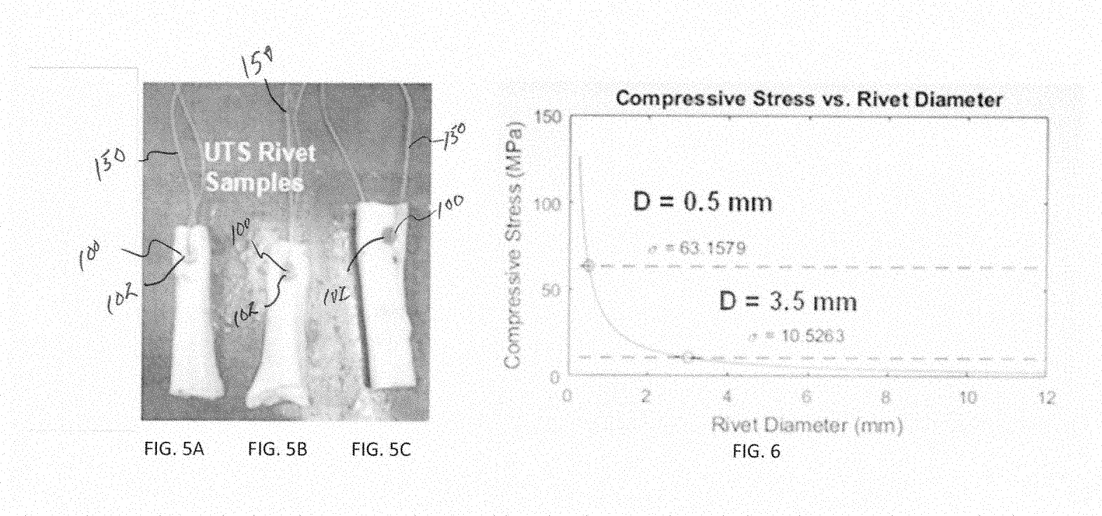

Referring to the graph of FIG. 6, modeling compressive stress vs. rivet diameter shows that increasing the diameter of the tendon-puncturing unit (i.e. suture or rivet) to 3.5 mm from 0.5 mm (the diameter of a #2 suture) results in a nearly 6-fold stress decrease in stress values. However, larger rivet diameters will also generate larger holes in the tendon, resulting in tendon weakening. Thus, there is some optimal rivet diameter where reduction of compressive stress is maximized while tendon weakening is minimized. This can be found by modeling the tendon weakening rate as the diameter of rivet 102 increases.

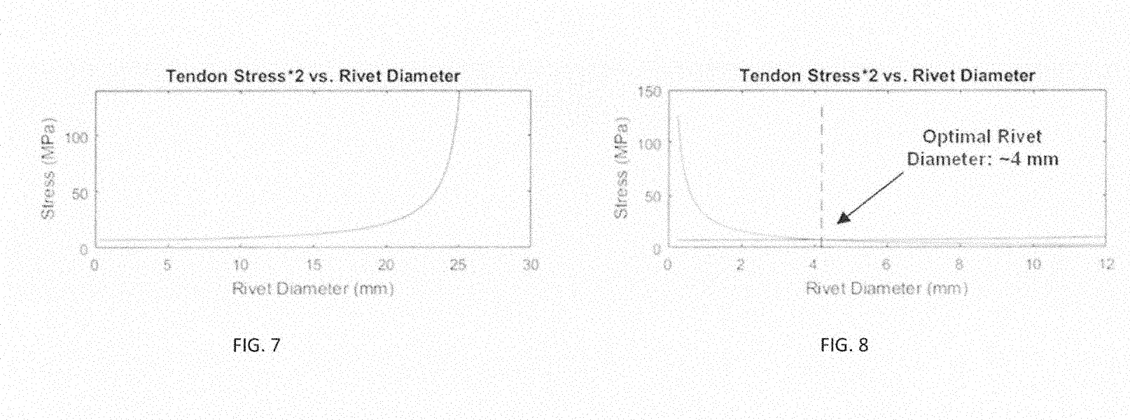

FIG. 7 shows the tendon stress, multiplied by a safety factor of 2, vs. rivet diameter. It can be seen that, for a rivet diameter of less than about 10, the stress is generally the same. By overlaying FIG. 6 onto FIG. 7 to find the optimal rivet diameter, FIG. 8 shows that an optimal diameter is about 4.2 mm. Therefore, a rivet 102 having female free end 112 with an outer diameter of about 4 millimeters is selected for use.

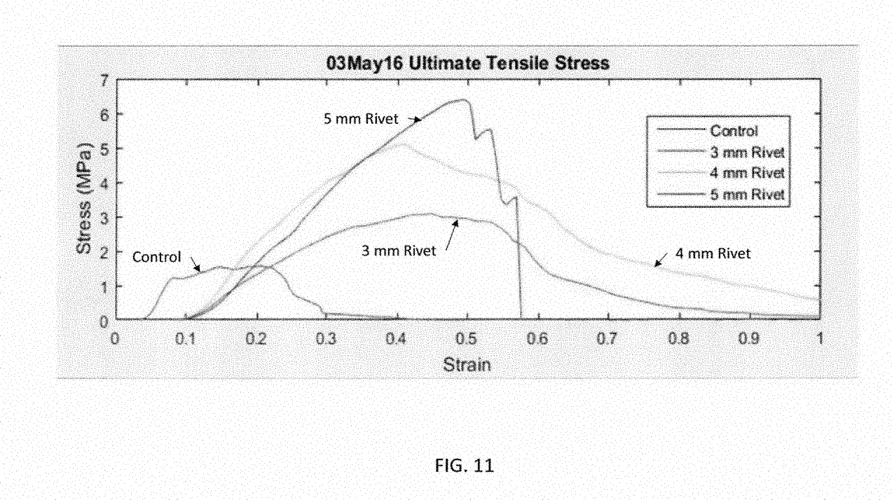

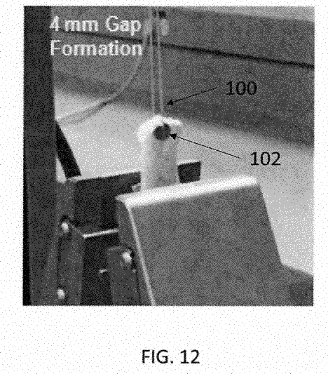

In tests that were performed using rivets 102 having 3 mm, 4 mm, and 5 mm diameters that were inserted into a bovine Achilles tendon, the testing revealed that the 3 mm, 4 mm, and 5 mm rivets increased the Ultimate Tensile Strength of the suture/tendon construct by approximately 100, 225, and 300%, respectively, compared to a control suture-only tendon. See FIG. 11. In all tests except the 5 mm rivet sample, failure occurred by the suture tearing through the tendon. For the 5 mm rivet sample, the suture snapped at the suture holder. Because the construct did not fail at the suture-tendon interface, the maximum stress can be considered to be an underestimate. In all of the rivet constructs, it is important to note there was a distinct gap formation that occurred in the tendon and grew as the test was performed. See FIG. 12.

It will be further understood that various changes in the details, materials, and arrangements of the parts which have been described and illustrated in order to explain the nature of this invention may be made by those skilled in the art without departing from the scope of the invention as expressed in the following claims.

We claim:

1. A tendon reinforcement assembly comprising:
a rivet comprising:
a female portion having a female free end, a female connecting end, and a female portion axial passage extending therethrough; and
a male portion having a male free end, a male connecting end adapted to be inserted into the female free end, and a male portion axial passage extending therethrough, the male portion axial passage extending coaxially with the female portion axial passage; and
a suture extending through the male portion axial passage and the female portion axial passage, the suture having a first suture end extending outwardly of the male free end and a second suture end extending outwardly of the female free end.

2. The tendon reinforcement assembly according to claim 1, wherein the male free end comprises a male flange.

3. The tendon replacement assembly according to claim 2, wherein the male flange comprises a beveled interior.

4. The tendon reinforcement assembly according to claim 1, wherein the female end comprises a female flange.

5. The tendon reinforcement assembly according to claim 1, wherein the female flange comprises a beveled interior.

6. The tendon reinforcement assembly according to claim 1, wherein the male portion and the female portion are constructed form biocompatible materials.

7. The tendon reinforcement assembly according to claim 1, wherein the female free end has an outer diameter of about 4 millimeters.

8. The tendon reinforcement assembly according to claim 1, wherein the rivet is adapted to be inserted into a rotator cuff.

9. A tendon reinforcement assembly comprising:
a rivet having an axial passage extending therethrough; and
a suture extending through the axial passage.

10. The tendon reinforcement assembly according to claim 9, wherein the rivet comprises a female portion and a male portion inserted into the female portion.

11. The tendon reinforcement assembly according to claim 9, wherein the rivet comprises a plurality of portions.

12. The tendon reinforcement assembly according to claim 9, wherein the rivet comprises a first flange at a first end of the axial passage and a second flange at a second end of the axial passage.

13. The tendon reinforcement assembly according to claim 12, wherein the axial passage comprises a curved transition to each of the first and second flanges.

14. The tendon reinforcement assembly according to claim 12, wherein the rivet has a body extending between the first flange and the second flange, wherein the body has a diameter of about 4 millimeters.

15. The tendon reinforcement assembly according to claim 12, wherein each of the first flange and the second flange is about 1 millimeter thick.

16. The tendon reinforcement assembly according to claim 12, wherein each of the first flange and the second flange is about 5 millimeters in diameter.

17. The tendon reinforcement assembly according to claim 9, wherein the axial passage is about 1 millimeter in diameter.

18. A tendon reinforcement assembly comprising:
a rivet comprising:
a female portion; and
a male portion inserted into the female portion,
wherein the rivet has an axial passage extending through both the female portion and the male portion.

19. The tendon reinforcement assembly according to claim 18, further comprising a suture passing through the axial passage.

20. The tendon reinforcement assembly according to claim 18, wherein the axial passage comprises a curved transition to a first flange at a female end of the female portion and to a second flange at a male end of the male portion.
